(19) United States
(12) Patent Application Publication
(10) Pub. No.: US 2012/0039844 A1
(43) Pub. Date: Feb. 16, 2012

(54) COMPOSITION AND USE OF N-ALPHA-METHYLHISTAMINE DIHYDROCHLORIDE FOR THE REDUCTION OF OXYGEN RADICAL FORMATION

(76) Inventor: Kristoffer Hellstrand, Goteborg (SE)

(21) Appl. No.: 13/264,321

(22) PCT Filed: Apr. 9, 2010
(86) PCT No.: PCT/US2010/030604

§ 371 Date: Oct. 13, 2011

Related U.S. Application Data
(60) Provisional application No. 61/169,992, filed on Apr. 16, 2009.

(51) Int. Cl. A61K 31/417; A61K 38/21; A61K 38/19; A61P 31/12; A61P 29/00; A61P 9/10; A61P 35/00; A61P 25/28; A61K 38/20; A61P 37/06

(52) U.S. Cl. 424/85.2; 424/85.7; 424/85.6; 424/85.5; 514/7.6; 514/400

(57) ABSTRACT

An improved composition and method for reducing the formation of oxygen radicals in mononuclear phagocytes is disclosed. Also provided is a method of protecting NK cells from oxidative damage inflicted by radicals produced by mononuclear phagocytes by administering a histamine metabolite, N-alpha-methylhistamine dihydrochloride. Use of N-alpha-methylhistamine dihydrochloride for enhancing the NK cell response to IL-2 is likewise detailed.

### BACKGROUND OF THE INVENTION

1. Field of the Invention

The disclosure below relates to the novel use of a histamine metabolite for reducing oxygen radical formation. More specifically, the histamine metabolite is particularly useful in reducing the formation of oxygen radicals in mononuclear phagocytes, protecting natural killer (NK cells) from oxidative damage, and enhancing the NK cell response to IL-2. Also provided are methods of treating conditions caused or exacerbated by oxygen radical formation. The histamine metabolite may be administered alone or in combination with additional agents. The additional agent may be an agent that stimulates the cytotoxic activity of NK cells and cytotoxic T lymphocytes (CTLs) in a synergistic fashion with the histamine metabolite.

2. Description of the Related Art

Recent anti-cancer and anti-viral strategies have focused on utilizing the host immune system as a means of cancer or antiviral treatment or therapy. The immune system has evolved complex mechanisms for recognizing and destroying foreign cells or organisms present in the body of the host. Harnessing the body's immune mechanisms is an attractive approach to achieving effective treatment of malignancies and viral infections.

A wide array of effector cells, each having its own characteristics and role, implement the immune response. One type of effector cell, the B cell, generates antibodies targeted against foreign antigens encountered by the host. In combination with the complement system, antibodies direct the destruction of cells or organisms bearing the targeted antigen.

Another type of effector cell, the T cell, is divided into subcategories which play different roles in the immune response. Helper T cells secrete cytokines which stimulate the functions of other cells necessary for mounting an effective immune response, while other T cells (“regulatory” T cells) down regulate the immune response. A third category of T cell, the cytotoxic T cell (CTL), is capable of directly lysing a targeted cell presenting a foreign antigen on its surface.

An additional type of effector cell is the natural killer cell (NK cell), a type of lymphocyte having the capacity to spontaneously recognize and destroy a variety of malignant and virus-infected cell types. This characteristic of NK cells makes them an attractive candidate for exploitation in anticancer and antiviral treatments and therapies based on using the host's immune system as a weapon against malignant tumors and viruses.

The interplay between the various effector cells listed above is influenced by the activities of a wide variety of chemical factors which serve to enhance or reduce the immune response as needed. Such chemical modulators may be produced by the effector cells themselves and may influence the activity of immune cells of the same or different type as the factor producing cell.

One category of chemical mediators of the immune response is cytokines, which are endogenous molecules that regulate functions of the cellular components of the immune system. Interleukin-2 (IL-2) is a cytokine synthesized by T cells that was first identified in conjunction with its role in the expansion of T cells in response to an antigen (Smith, K. A. Science 240:1169 (1988). It is well known that IL-2 secretion is necessary for the full development of cytotoxic effector T cells (CTLs), which play an important role in the host defense against viruses and malignant cells. Several studies have also demonstrated that IL-2 has antitumor effects that make it an attractive agent for treating malignancies (see e.g. Lotze, M. T. et al, in “Interleukin 2”, ed. K. A. Smith, Academic Press, Inc., San Diego, Calif., p 237 (1988); Rosenberg, S., Ann. Surgery 208:121 (1988)). In fact, IL-2 has been utilized to treat subjects suffering from malignant melanoma, renal cell carcinoma, and acute myelogenous leukemia. (Rosenberg, S. A. et al., N. Eng. J. Med. 316:889-897 (1978); Bukowski, R. M. et al., J. Clin. Oncol 7:477-485 (1989); Foa, R. et al., Br. J. Haematol. 77:491-496 (1990)).

It appears likely that NK cells are, at least to significant extent, responsible for the anti-tumor effects of IL-2. For example, IL-2 rapidly and effectively augments the cytotoxicity of isolated human NK cells in vitro (Dempsey, R. A., et al., J. Immunol. 129:2504 (1982); Phillips, J. H., et al. J. Exp. Med. 170:291 (1989)). Thus, the cytotoxic activity against malignant cells of NK cells treated with IL-2 is greater than the constitutive levels of cytotoxicity observed in untreated cells. Furthermore, depletion of NK cells from animals eliminates IL-2's antitumor effects in several models of experimental tumor growth. (Mule, J. J. et al, J. Immunol. Invest. 139:285 (1987); Lotze, M. T. et al., supra). Additional evidence for the role of NK cells results from the observation that NK cells are the only resting human peripheral blood lymphocytes expressing the IL-2 receptor on their cell surface. (Caliguri, M. A. et al., J. Clin. Invest. 91:123-132 (1993)).

Another cytokine with promise as an anti-cancer and antiviral agent is interferon-α. Interferon-α (IFN-α) has been employed to treat leukemia, myeloma, and renal cell carcinomas. Isolated NK cells exhibit enhanced cytotoxicity in the presence of IFN-α. Thus, like IL-2, IFN-α also acts to augment NK cell mediated cytotoxicity. (Trinchieri, G. Adv. Immunol. 47:187-376 (1989)).

Previously, we have demonstrated that the administration of histamine, a biogenic amine, acts synergistically with cytokines to augment the cytotoxicity of NK cells and to improve the function of CTLs. Thus, therapies employing the combination of histamine and cytokines represent an attractive approach to anti-cancer strategies based on using the host immune system to attack the malignancy.

Histamine's synergistic effect when combined with cytokines is not the result of a direct positive effect of histamine on NK cells and CTLs. Rather, the synergistic effects result from the suppression of a down regulation of functions of NK cells and CTLs that is mediated by other cell types present. The discussion below provides some of the evidence suggesting that histamine's synergistic effects result from the suppression of negative signals exerted by other cell types.

U.S. Pat. No. 5,348,739, which is incorporated herein by reference, discloses the synergistic effects of histamine and interleukin-2. As discussed above, IL-2 normally induces a cytotoxic response in NK cells. In vitro studies with NK cells alone confirm that cytotoxicity is stimulated when IL-2 is administered. However, the IL-2 induced enhancement of cytotoxicity of NK cells is suppressed in the presence of mononuclear phagocytes, which are myeloid cells abundantly present in blood (monocytes) and tissues (macrophages).

In the absence of monocytes, histamine had no effect or weakly suppressed NK mediated cytotoxicity. (U.S. Pat. No. 5,348,739; Hellstrand, K. et al., J. Immunol. 137:656 (1986); Hellstrand, K. and Hermodsson, S., Int. Arch. Allergy Appl. Immunol. 92:379-389 (1990)). However, NK cells exposed to histamine and IL-2 in the presence of monocytes exhibit elevated levels of cytotoxicity relative to that obtained when NK cells are exposed only to IL-2 in the presence of monocytes. Id. Thus, the synergistic enhancement of NK cell cytotoxicity by combined histamine and interleukin-2 treatment results not from the direct action of histamine on NK cells but rather from suppression of an inhibitory signal generated by monocytes. Specifically, histamine activates the H2 histamine receptor on monocytes to improve NK cell cytotoxicity and the NK cell response to IL-2 (Hellstrand et al., J. Immunol. 1994 Dec. 1; 153(11):4940-7). Without being limited to a particular mechanism, it is believed that the inhibitory effects of monocytes on cytotoxic effector cells such as NK cells and CTLs result from the generation of H₂O₂ by monocytes. H₂O₂ and other reactive oxygen species (ROS) are formed by monocytes and other myeloid cells as part of the innate immune defense against bacteria and parasites (cf. below). However, ROS may also damage neighboring cells, including lymphocytes. It has been reported that the production of H₂O₂ by monocytes suppresses NK cell cytotoxicity. (Van Kessel, K. P. M. et al., Immunology, 58:291-296 (1986); El-Hag, A. and Clark, R. A. J. Immuol. 133:3291-3297 (1984); Seaman, W. E. et al., J. Clin. Invest. 69:876-888 (1982)). Further evidence of the role of H₂O₂ in suppressing NK cell functions comes from in vitro studies showing that the addition of catalase, an enzyme which acts to degrade H₂O₂, to preparations of monocytes and NK cells exposed to IL-2 removes the inhibitory effects of the monocytes. (Seaman, supra.)

It has been suggested that histamine, acting via the H2 receptor, may exert its synergistic effects by reducing the level of H₂O₂ produced by monocytes. Hellstrand, K., Asea, A., Hermodsson, S. Histaminergic regulation of antibody-dependent cellular cytotoxicity of granulocytes, monocytes and natural killer cells, J. Leukoc. Biol. 55:392-397 (1994). Hellstrand et al., J. Immunol. 1994, 152: 4940-4947

Monocytes are not the only cell type which negatively regulates NK cell and CTL functions. Experiments have demonstrated that granulocytes suppress both the constitutive and IL-2 induced cytotoxic activity of NK cells in vitro. Like the monocyte mediated suppression discussed above, granulocyte mediated suppression is synergistically overcome by treatment with IL-2 and histamine. (U.S. Pat. No. 5,348,739; Hellstrand, K., Asea, A., Hermodsson, S. Histaminergic regulation of antibody-dependent cellular cytotoxicity of granulocytes, monocytes and natural killer cells, J. Leukoc. Biol. 55:392-397 (1994).

Therapies employing histamine and cytokines are effective anti-cancer strategies. U.S. Pat. No. 5,348,739 discloses that mice given histamine and IL-2 prior to inoculation with melanoma cell lines were protected against the development of lung metastatic foci. This effect was a consequence of synergistic interaction between histamine and IL-2, as demonstrated by the significant reduction in metastatic foci observed in mice given histamine and IL-2 as compared to mice given histamine or IL-2 alone.

In addition to the synergistic effects observed in the assay of lung metastatic foci, synergistic effects of histamine plus IL-2 treatment were also observed in assays in which NK cell cytotoxicity was measured by determining the ability of mice to kill malignant cell lines derived from both humans and mice which were injected into them. Id. Moreover, in studies conducted to investigate the role of histamine in NK-cell dependent protection against herpes simplex virus (HSV) type 2, it was discovered that a single dose of histamine could prolong survival time in animals inoculated intravenously with HSV, and a synergistic effect on the survival time of animals treated with a combination of histamine and IL-2 was observed (Hellstrand, K. et al., Role of histamine in natural killer cell-dependent protection against herpes simplex virus type 2 infection in mice., Clin. Diagn. Lab. Immunol. 2:277-280 (1995)).

Strategies employing a combination of histamine and IL-2 are an effective means of treating malignancies and viral infection. Histamine also acts synergistically with interferon-α to overcome the suppression of NK cell cytotoxicity by monocytes (Hellstrand et al., Regulation of the NK cell response to interferon-α by biogenic amines, J. Interferon Res. 12:199-206 (1992)). Like IL-2, interferon-α augments NK cell constitutive NK cell cytotoxicity. Id. Monocytes suppress the interferon-α mediated enhancement of NK cell killing of malignant target cells in vitro. Monocyte mediated suppression of NK cell cytotoxicity was overcome by treatment with histamine and interferon-α.

The in vitro and animal results discussed above suggested that histamine+IL-2, histamine+interferon-α or histamine+IL-2+interferon-α were promising methods for treating human malignancies. In fact, combinations of histamine and IL-2 treatments have proven effective in the treatment of human malignancies, providing a response rate significantly greater than that observed with IL-2 alone. Agarwala et al., J Clin Oncol 2002 Jan. 1; 20(1):125-33 (a phase III trial in melanoma) thus reported a significant survival superiority of treatment with histamine/IL-2 vs. IL-2 in 129 patients with liver melanoma. Donskov et al., Br J Cancer. 2005 Oct. 3; 93(7):757-62 reported a significant survival superiority of histamine/IL-2 vs. IL-2 in a randomized phase II trial of 63 patients with metastatic renal cell carcinoma. Brune et al., 2006 Jul. 1; 108(1):88-96, reported in a phase III trial that histamine/IL-2, when compared with the current standard of care, significantly prevented leukemic relapse in 320 patients with acute myeloid leukemia, whereas IL-2 as the single agent has been inefficacious (see e.g. Baer et al., J Clin Oncol 2008 Oct. 20; 26(30):4934-9; Blaise et al., Eur Cytokine Netw. 2000 March; 11(1):91-8).

While histamine alone or in combination with other therapeutic agents has been a great boon to medicine, the use of histamine is not without health risk. Histamine activates the H1, H2, H3 and H4 histamine receptors and thus exerts a wide array of biological responses, some of which can be toxic to the recipient. The inflammatory response triggered by histamine can result in a number of untoward side effects which range in severity from abdominal or stomach spasms or cramps to anaphylaxis.

There remains a need to discover other agents which augment a patient's immune response to eliminate a substantial amount of human suffering while minimizing negative side effects.

### SUMMARY OF THE INVENTION

The present invention relates to the novel use of a histamine metabolite for reducing oxygen radical formation and therapeutic indications associated with such reduction. This histamine metabolite is particularly useful in reducing the formation of oxygen radicals in mononuclear phagocytes, protecting NK cells from oxidative damage, and enhancing the NK cell response to IL-2.

In one aspect of the invention, an improved method of augmenting NK cell cytotoxicity with a histamine metabolite while reducing untoward side effects associated with histamine administration is provided. The method includes administering a pharmaceutically effective amount of N-alpha-methylhistamine dihydrochloride and a cytokine. The cytokine may be interleukin-2. Alternatively, or in addition to, the cytokine may be Interferon-α, Interferon-β, Interferon-γ, IL-1, IL-3, IL-12, IL-15, or combinations thereof.

In another aspect of the invention, the effective amount of N-alpha-methylhistamine dihydrochloride is between about 0.005 and 10 mg/kg/day. Optionally, the cytokine is administered in an amount of between about 5,000 and 500,000 U/kg/day.

In still another aspect of the invention, a method of enhancing the NK cell response to IL-2 is provided. The method includes administering to a population of NK cells a pharmaceutically effective amount of IL-2 and a pharmaceutically effective amount of N-alpha-methyl-histamine dihydrochloride. Advantageously, the pharmaceutically effective amount of IL-2 is between about 5,000 and 500,000 U/kg/day and the pharmaceutically effective amount of N-alpha-methyl-histamine dihydrochloride is between about 0.005 and 10 mg/kg/day.

In yet another aspect of the invention, a method of treating a condition caused or exacerbated by oxygen radical formation is described. The method includes identifying an individual suffering from a condition caused or exacerbated by oxygen radical formation; and administering to the individual a pharmaceutically effective amount of N-alpha-methyl-histamine dihydrochloride.

Optionally, the condition caused or exacerbated by oxygen radical formation is a viral infection, autoimmune disease, inflammatory disease, atherosclerosis, cancer, or neurodegenerative disease. Advantageously, the effective amount of N-alpha-methyl-histamine dihydrochloride is between about 0.005 and 10 mg/kg/day.

In yet another aspect of the invention, a method of protecting NK cells from oxidative damage inflicted by oxygen radicals in a subject, for the treatment of cancer, viral diseases, or inflammatory diseases is provided. The method includes identifying a subject in need of NK cell enhancement and administering to the subject an effective amount of N-alpha-methyl-histamine dihydrochloride. Optionally, the method further includes administering a cytokine such as IL-1, IL-2, IL3, IL12, IL-15, IFN-α, IFN-β, or IFN-γ.

In still another aspect of the invention, a use of N-alpha-methyl-histamine dihydrochloride for the manufacture of a medicament for treating a condition caused or exacerbated by oxygen free radical formation is provided. The condition treated may be a viral infection, autoimmune disease, inflammatory disease, atherosclerosis, cancer, or a neurodegenerative disease.

In yet another aspect of the invention, a compound comprising a pharmaceutically effective amount of N-alpha-methyl-histamine dihydrochloride along with a cytokine is provided.

### BRIEF DESCRIPTION OF THE DRAWINGS

FIG. 1 is a graphical representation of the effect of N-α-methylhistamine (N-α-MH) on fMLF induced respiratory burst.

FIG. 2 is a graph illustrating the effectiveness of N-α-methylhistamine in protecting NK cells against oxidatively induced apoptosis.

FIG. 3 is a graph depicting CD69 expression in NK cells exposed to IL-2 and histamine or N-α-methylhistamine.

FIG. 4 is a graph illustrating the effectiveness of N-α-methylhistamine as compared with histamine in preventing tumor formation in mice inoculated with B16 melanoma cells.

FIG. 5 is another graph illustrating the effectiveness of N-α-methylhistamine as compared with histamine in preventing tumor formation in mice inoculated with B16 melanoma cells.

### DETAILED DESCRIPTION OF THE PREFERRED EMBODIMENT

The disclosure below relates to a composition and methods for preventing and reducing cellular and tissue damage caused by reactive oxygen species (ROS). More particularly, a histamine metabolite is provided which more potent than histamine dihydrochloride at histamine H2 receptors (Saitoh et al, 2002, referenced below) but a weak agonist at histamine H1 receptors (White et al., Br J Pharmacol. 1993 vol. 108(1): 196-203) and thus likely to cause significantly less untoward side effects than histamine dihydrochloride. The metabolite is useful in reducing the formation of oxygen radicals in mononuclear phagocytes, protecting NK cells from oxidative damage inflicted by radicals produced by mononuclear phagocytes, and enhancing the NK cell response to IL-2.

The effects of ROS production are many faceted. ROS are known to cause apoptosis in NK cells. ROS are also known to cause anergy and/or apoptosis in T-cells. The mechanisms by which ROS cause these effects are not yet fully understood. Nevertheless, some commentators believe that ROS cause cell death by disrupting cellular membranes and by changing the pH of cellular pathways critical for cell survival and also by direct damaging effects on DNA.

Reactive oxygen metabolites are often produced by the incomplete reduction of oxygen. The complete reduction of one molecule of O₂ to water is a four-electron process. Oxidative metabolism continually generates partially reduced species of oxygen, which are far more reactive, and hence more toxic than O₂ itself. A one-electron reduction of O₂ yields superoxide ion (O₂⁻); reduction by an additional electron yields hydrogen peroxide (H₂O₂), and reduction by a third electron yields a hydroxyl radical (OH.), and a hydroxide ion. Nitrous oxide (NO), is another interesting reactive oxygen metabolite, produced through an alternative pathway. Hydroxyl radicals in particular are extremely reactive and represent the most active mutagen derived from ionizing radiation. All of these species are generated and must be converted to less reactive compounds to avoid tissue damage.

As discussed above, particular cells of the immune system have harnessed the toxic effects of ROS as an effector mechanism. Phagocytic cells of the myeloid lineage such as polymorphonuclear leukocytes (neutrophils, PMN), monocytes, macrophages, and eosinophils function to protect the host in which they reside from infection by seeking out and destroying invading microbes. These phagocytic cells possess a membrane-bound enzyme system, the NADPH oxidase, which can be activated to produce toxic oxygen radicals in response to a wide variety of stimuli.

The “increased respiration of phagocytosis” (the respiratory burst) was reported and thought to be a result of increased mitochondrial activity providing additional energy for the processes of phagocytosis. It was later shown that a non-mitochondrial enzymatic system produced the increased levels of oxygen metabolites since the respiratory burst continued even in the presence of mitochondrial inhibitors such as cyanide and antimycin A. In 1968, Paul and Sbarra showed clearly that stimulated phagocytes produced hydrogen peroxide and in 1973 Babior and co-workers established that superoxide was a major product of the oxidase. See Paul and Sbarra, Biochim Biophys Acta 156(1): 168-78 (1968); Babior, et al., J Clin Invest 52(3): 741-4 (1973). It is now generally accepted that the enzyme is membrane bound, exhibits a preference for NADPH (Kₘ=45 μM) over NADH (Kₘ=450 μM), and converts oxygen to its one electron-reduced product, superoxide.

NADPH+H⁺+2O₂→NADP⁺+2H⁺+2O₂⁻

The hydrogen peroxide arises from subsequent dismutation of the superoxide.

2O₂⁻+2H⁺→H₂O₂+O₂⁻

The enzyme activity is barely detectable in resting (unstimulated) phagocytes, but increases dramatically upon stimulation. Neutrophils and macrophages produce oxidizing agents to break through the protective coats or other factors that protect phagocytosed bacteria. The large quantities of superoxide, hydrogen peroxide, and hydroxyl ions are all lethal to many bacteria, even when found in very small quantities. While there are beneficial effects of these oxygen metabolites, it is clear that inappropriate production of oxygen metabolites can result in severely deleterious effects.

N-alpha-methyl-histamine (N-α-MH) is a potent histamine H₂ and H₃ receptor agonist having the following chemical structure:

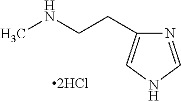

N-α-MH is significantly more potent than histamine in terms of stimulating cAMP production via the H₂ receptor with a lower EC₅₀ value and higher maximal cAMP production See T. Saitoh et al., Gut 2002; 50: 786-789. In addition to increased potency in cAMP production, N-α-MH appears at least as efficacious at inhibiting oxygen radical production than histamine dihydrochloride. As will be described in greater detail below, N-α-MH efficiently inhibits oxygen radical production. The maximal response in terms of inhibition of oxygen radical production is also stronger using N-α-MH than histamine dihydrochloride. N-α-MH also protects NK cells and synergizes with IL-2 to activate NK cells.

N-α-MH offers many further clinical and therapeutic advantages over histamine dihydrochloride. Specifically, N-α-MH is expected to have a significantly longer half life than histamine dihydrochloride. Histamine dihydrochloride has a relatively short half life, on the order of five minutes, in the blood. Beaven. M. A., Factors regulating availability of histamine at tissue receptors in Pharmacology of Histamine Receptors, C. R. Ganellin and M. E. Parsons eds. Wright PSG. Bristol, U.K. pp. 103-145 (1982). By contrast, N-methylated derivatives of histamine typically have a half life that exceeds that of histamine See, e.g. Interdependence of histamine and methylhistamine kinetics: modeling and simulation approach Computers in Biology and Medicine, Volume 29, Issue 6, Pages 361-375). Because N-α-MH may have a longer half life than histamine dihydrochloride, sustained, stable therapeutic levels of N-α-MH in an individual's blood may be achieved more readily and for longer periods of time than with histamine dihydrochloride.

Moreover, while histamine potently activates H1, H2, and H3 histamine receptors, N-α-MH has known significant intrinsic activity only at H2 and H3 receptors (See, generally Saitoh, T et al. in Gut and White et al, referenced above; See, also Korte, A. et al. 168 Biochem Biophys Res Commun: pp. 979-986 (1990); Chiavegatto, S. et al. 62 Life Sci pp. 1875-1888 (1998); Kathmann, M. et al., 358 Naunyn Schmiedebergs Arch Pharmacol. pp. 623-627 (1998); West, R. E. et al., 377 Eur J Pharmacol pp. 233-239 (1999); and Murray, S. et al. 739 J Chromatogr B Biomed Sci Appl pp. 337-344 (2000)), meaning that N-α-MH is likely to produce less side-effects than histamine. It is believed that intermittent dosing of N-α-MH is substantially less toxic than histamine because N-α-MH is more specific than histamine. Histamine dihydrochloride use has been associated with a number of side effects including, without limitation, discomfort, diarrhea, nausea or vomiting, stomach pain, swelling or redness at the place of injection, dizziness, light-headedness, fainting, fast or pounding heartbeat, headache, nervousness, seizures, difficulty in breathing, pronounced flushing or redness, blurred vision, chest discomfort or pain, bronchospasm, hypotension, sudden decrease in blood pressure, severe diarrhea, difficulty in breathing, heart failure, and possibly death. By contrast, N-α-MH can be utilized for clinical therapeutics without the same severity of side effects since N-α-MH has little or no activity at H1 receptors, which translates into less toxicity.

A method of reducing the formation of oxygen radicals in mononuclear phagocytes by administering N-α-MH is provided. The invention disclosed herein is based, in part, on the discovery that N-α-MH efficiently reduces the amount of ROS produced and released in an individual. In preferred embodiments, N-α-MH is used to achieve a beneficial reduction or inhibition of enzymatic ROS production and release or the net concentration thereof.

As will be described in greater detail below, N-α-MH can be administered alone to achieve a beneficial reduction or inhibition of enzymatic ROS or in combination with other beneficial agents such as compounds that induce the release of endogenous histamine from a patient's own tissue stores is also included within the scope of the present disclosure. Such compounds include IL-3, retinoids, and allergens. Other ROS production and release inhibitory compounds such as NADPH oxidase inhibitors like diphenyleneiodonium can also be used in combination with N-α-MH with the disclosed methods, as can serotonin and 5HT-receptor agonists. The compounds of the present invention can be administered separately or as a single composition (combined). If administered separately, the compounds should be given in a temporally proximate manner, e.g. within a twenty-four hour period, such that the reduction or inhibition of enzymatic ROS is enhanced. More particularly, the compounds may be given within one hour of each other. The administration can be by either local or by systemic injection or infusion. Other methods of administration may also be suitable

The compositions and methods disclosed herein also encompass the administration of N-α-MH in combination with a variety of ROS scavengers. Known scavengers of ROS include the enzymes catalase, superoxide dismutase (SOD), glutathione peroxidase and ascorbate peroxidase. Additionally, vitamins A, E, and C are known to have scavenger activity. Minerals such as selenium and manganese can also be efficacious in combating ROS-mediated damage. The scope of the methods disclosed herein includes the administration of the compounds listed and those compounds with similar ROS inhibitor activity. The compositions and methods disclosed herein also provide an effective tool for preventing and/or inhibiting the release of enzymatically generated ROS in excessive amounts or at inappropriate times or locations.

Compounds and methods for treating disease states that are complicated by the detrimental release of ROS within a host or subject are provided comprising the administration of a pharmaceutically effective amount of N-α-MH alone or in combination with other therapeutic agents. Conditions caused or exacerbated by enzymatically produced ROS-mediated oxidative damage are well known in the art. (See U.S. Pat. No. 6,242,473, hereby incorporated by reference in its entirety.) The conditions contemplated as treatable under the present invention result from disparate number of etiological causes. Nevertheless, they share a common feature in that their pathological conditions are either caused or exacerbated by enzymatically produced ROS-mediated oxidative damage caused by inappropriate and harmful concentrations of ROS. The present invention contemplates the administration of N-α-MH to treat conditions where ROS play an active and detrimental role. Exemplary conditions include but are not limited to: Adult Respiratory Distress Syndrome (ARDS); chronic obstructive pulmonary disease (COPD), meningitis, ischemia/reperfusion injury such as stroke, myocardial infarction, atherosclerosis, complications of mechanical ventilation or septic shock; treatment of infectious diseases such as hepatitis C, acquired immunodeficiency syndrome (AIDS), or herpes virus infection; various autoimmune or inflammatory disorders where ROS are believed to play a detrimental role such as multiple sclerosis (MS) and rheumatoid arthritis, and Inflammatory Bowel Diseases such as Crohn's disease and ulcerative colitis; various neurodegenerative disease where ROS are thought to contribute to the disease state, such as ALS, Alzheimer's disease, and Parkinson's disease; as well as other clinical conditions wherein enzymatically produced ROS can play an important role such as in radiation injury and cancer. Thus, the administration of N-α-MH, alone or in combination with other beneficial compounds, provides an effective treatment for a variety of medical conditions.

A method of protecting NK cells from oxidative damage inflicted by radicals produced by mononuclear phagocytes is similarly provided. The method includes administering to a subject in need thereof a pharmaceutically effective amount of N-α-MH. Also disclosed herein is an improved method for the prevention of the inactivation of natural killer cells and the enhanced activation of NK cells in the presence of monocytes using a combination of an NK cell activator and N-α-MH. The method is useful, for example, in the treatment of solid tumors, metastases, and viral infection. Given that NK cells exert antibody-dependent cytotoxicity against malignant cells, N-α-MH can also be combined with antibodies used in the treatment of malignancies. Malignancies against which treatment with N-α-MH can be directed include, but are not limited to, primary and metastatic malignant solid tumor disease, and hematological malignancies. Exemplary cancers include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophogeal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas cystadenocarcinoma, medullary carcinoma, bronchogenic carcinoma, renal cell carcinoma, hepatoma bile duct carcinoma choriocarcinoma seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme astrocytoma medulloblastoma, craniopharyngioma, ependymoma, pinealoma, hemangioblastoma acoustic neuroma, oligodendroglioma, meningioma, skin cancer, melanoma, neuroblastoma, retinoblastoma, Blood-borne cancers, including but not limited to: acute lymphoblastic B-cell leukemia, acute lymphoblastic T-cell leukemia, acute myeloblastic leukemia, “AML,” acute promyelocytic leukemia “APL,” acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia, “CML,” chronic lymphocytic leukemia, “CLL,” hairy cell leukemia, multiple myeloma, lymphoblastic myelogenous leukemias, lymphocytic myelocytic leukemias, lymphomas: such as Hodgkin's disease, non-Hodgkin's Lymphoma, Multiple myeloma, Waldenstrom's macroglobulinemia, Heavy chain disease, myelodysplastic syndrome, and Polycythemia vera.

N-α-MH in combination with a cytokine or other NK cell activator serves to activate NK cells in the presence of monocytes, thereby providing a novel method for inhibiting tumor growth, interfering with the formation of metastases, and treating viral infections. As used herein, the term “cytokine” includes, without limitation, any number of agents which stimulate NK cell cytotoxicity in the presence of monocytes including interleukins, interferons, and flavanoids. Suitable interleukins include, for example, IL-1, IL-2, IL-12, and IL-15. Exemplary interferons for use with N-α-MH include, for example, IFN-α, IFN-β, and IFN-γ. Exemplary flavanoids for use in the present invention include, for example, favone-8-acetic acid (FAA) and xanthenone-4-acetic acid (XAA). Preferred dosage range can be determined using techniques known to those having ordinary skill in the art. The N-α-MH is administered in an amount of from about 0.1 to about 10 mg/day; more preferably, the amount is from about 0.5 to about 8 mg/day; and even more preferably, the amount is from about 1 to about 5 mg/day. Preferably, the interleukin is administered in an amount of from about 1,00 to about 500,000 U/kg/day; more preferably, the amount is from about 3,000 to about 100,000 U/kg/day, and even more preferably, the amount is from about 5,000 to about 20,000 U/kg/day. The interferons can be administered in an amount of from about 1,000 to about 300,000 U/kg/day, more preferably, the amount is from about 3,000 to about 100,000 Uk/kg/day, and even more preferably, the amount is from about 10,000 to about 50,000 U/kg/day. Flavanoid compounds can be administered in an amount of from about 1 to about 100,000 mg/day; more preferably, the amount is from about 5 to about 10,000 mg/day, and even more preferably, the amount is from about 50 to about 1,000 mg/day. The N-α-MH and NK cell-activator can be administered as a single formulation or administered separately. In one embodiment, the N-α-MH is administered first. In another embodiment, the NK cell-activator is administered first.

N-α-MH is a highly potent histamine metabolite which augments the anti-tumor reactivity of NK cells when administered alone or in combination with IL-2. N-α-MH activates NK cell cytotoxicity by a mechanism of action involving a cell-to-cell mediated interaction between monocytes and phenotypically distinct NK cells. IL-2, a T cell-derived lymphokine, effectively activates NK cells and exerts antitumor effects. It is believed that N-α-MH and IL-2 act synergistically to augment NK cell activity. Thus, in one embodiment, a method of inhibiting the development of malignant tumors and the formation of metastases of malignant tumor cells in a subject in need thereof is provided. The method includes the administration of N-α-MH in combination with IL-2. IL-2 and N-α-MH can be administered separately or in the same composition. The administration can be attained by routes which are known in the art for these compounds and preparations. By means of example, they can be administered by local or systemic injection, or infusion as is known in the art. Typically, the N-α-MH is administered in an amount of about 0.1 to 10 mg/day, preferably about 0.5 to 8 mg/day, and more preferably about 1 to 5 mg/day. However, other amounts can also be administered with IL-2 as can be tailored by the practitioner.

The IL-2 can be administered in an amount of about 1,000 to 500,000 U/kg/day, more preferably about 3,000 to 100,000 U/kg/day, and more preferably about 5,000 to 20,000 U/kg/day, or otherwise as known in the art.

A daily dose can be administered as one dose or it can be otherwise divided into several doses if negative effects are observed. In one preferred embodiment, the N-α-MH and IL-2 are administered on the same days. A still more preferred embodiment is one wherein the N-α-MH and IL-2 are administered in the same composition. In another aspect of the invention, it is provided herein a method of increasing the anti-tumor cell effect of IL-2 in a subject comprising co-administering to a subject a first composition comprising IL-2 and a second composition comprising N-α-MH; the N-α-MH and IL-2 being administered in amounts and for a period of time effective to attain the desired effect. The N-α-MH can be administered separately from the IL-2 or as a single formulation. In one embodiment, the N-α-MH is administered first. In another embodiment, the IL-2 is administered first.

The use of the N-α-MH can be by any of a number of methods well known to those of skill in the art. For oral administration, the N-α-MH be incorporated into a tablet, aqueous or oil suspension, dispersible powder or granule, microbead, emulsion, hard or soft capsule, syrup or elixir. The compositions can be prepared according to any method known in the art for the manufacture of pharmaceutically acceptable compositions and such compositions can contain one or more of the following agents: sweeteners, flavoring agents, coloring agents and preservatives. Tablets containing the active ingredients in admixture with non-toxic pharmaceutically acceptable excipients suitable for tablet manufacture are acceptable. “Pharmaceutically acceptable” means that the agent should be acceptable in the sense of being compatible with the other ingredients of the formulation (as well as non-injurious to the individual). Such excipients include inert diluents such as calcium carbonate, sodium carbonate, lactose, calcium phosphate or sodium phosphate; granulating and disintegrating agents, such as corn starch and alginic acid; binding agents such as starch, gelatin or acacia; and lubricating agents such as magnesium stearate, stearic acid or talc. Tablets may be uncoated or may be coated with known techniques to delay disintegration and absorption in the gastrointestinal tract and thereby provide a sustained action over a longer period of time. For example, a time delay material such as glyceryl monostearate or glyceryl stearate alone or with a wax may be employed.

In another preferred embodiment, tablets, capsules or microbeads are coated with an enteric coating which prevents dissolution in the acidic environment of the stomach. Instead, this coating dissolves in the small intestine at a more neutral pH. Such enteric coated compositions are described by Bauer et al., Coated Pharmaceutical Dosage Forms: Fundamentals, Manufacturing Techniques, Biopharmaceutical Aspects, Test Methods and Raw Materials, CRC Press, Washington, D.C., 1998, the entire contents of which are hereby incorporated by reference.

Formulations for oral use can also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil.

Aqueous suspensions contain N-α-MH in admixture with excipients for the manufacture of aqueous suspensions. Such excipients include suspending agents, dispersing or wetting agents, one or more preservatives, one or more coloring agents, one or more flavoring agents and one or more sweetening agents such as sucrose or saccharin.

Oil suspensions can be formulated by suspending the active ingredient in a vegetable oil, such as arachis oil, olive oil, sesame oil or coconut oil, or in a mineral oil such as liquid paraffin. Optionally, the oil suspension contains a thickening agent, such as beeswax, hard paraffin or cetyl alcohol. Sweetening agents, such as those set forth above, and flavoring agents can also be added to provide a palatable oral preparation. These compositions can be preserved by an added antioxidant such as ascorbic acid. Dispersible powders and granules of the compounds of the invention, suitable for preparation of an aqueous suspension by the addition of water, provide the active ingredient in admixture with a dispersing or wetting agent, a suspending agent, and one or more preservatives. Additional excipients, for example sweetening, flavoring and coloring agents, can also be present.

Syrups and elixirs are formulated with sweetening agents, such as glycerol, sorbitol or sucrose. Such formulations may also contain a demulcent, a preservative, a flavoring or a coloring agent.

The use of compounds comprising N-α-MH can also be accomplished via parenteral delivery through subcutaneous, intravenous, intraperitoneal, or intramuscular injection. The compounds can be administered in an aqueous solution with or without a surfactant such as hydroxypropyl cellulose. Dispersions are also contemplated such as those utilizing glycerol, liquid polyethylene glycols, and oils. Injectable preparations can include sterile aqueous solutions or dispersions and powders that can be diluted or suspended in a sterile environment prior to use. Carriers such as solvents or dispersion media contain water, ethanol polyols, vegetable oils and the like can also be added to the disclosed compounds. Coatings such as lecithins and surfactants can be used to maintain the proper fluidity of the composition. Isotonic agents such as sugars or sodium chloride can be added, as well as products intended to delay absorption of the active compounds such as aluminum monostearate and gelatin. Sterile injectable solutions are prepared according to methods well known to those of skill in the art and can be filtered prior to storage and/or use. Sterile powders can be vacuum or freeze dried from a solution or suspension. Sustained or controlled release preparations and formulations can also be used with the disclosed methods. Typically the materials used with the disclosed methods and compositions are pharmaceutically acceptable and substantially non-toxic in the amounts employed.

The disclosed compounds can also be administered by inhalation. In this administration route, N-α-MH is dissolved in water or some other pharmaceutically acceptable carrier liquid for inhalation, or provided as a dry powder, and then introduced into a gas or powder that is then inhaled by the patient in an appropriate volume so as to provide that patient with a measured amount of histamine. Examples of the administration of a therapeutic composition via inhalation are described in U.S. Pat. Nos. 6,418,926; 6,387,394; 6,298,847; 6,182,655; 6,132,394; and 6,123,936, which are hereby incorporated by reference.

Infusion devices can be used to deliver the disclosed compounds. Suitable devices include syringe pumps, auto injector systems, implantable pumps, implantable devices, and minipumps. Exemplary devices include the Ambulatory Infusion Pump Drive, Model 30, available from Microject Corp., Salt Lake City, Utah, and the Baxa Syringe Infuser, available from Baxa Corporation, Englewood, Colo. Any device capable of delivering the disclosed compounds in accordance with the methods disclosed herein can be used.

Suitable infusion devices preferably have an effective amount of N-α-MH. The device can be pre-loaded with the desired substance during manufacture, or the device can be filled with the substance just prior to use. Pre-filled infusion pumps and syringe pumps are well known to those of skill in the art. The active substance can be part of a formulation which includes a controlled release carrier, if desired. A controller is used with the device to control the rate of administration and the amount of substance to be administered. The controller can be integral with the device or it can be a separate entity. It can be pre-set during manufacture, or set by the user just prior to use. Such controllers and their use with infusion devices are well known to those of skill in the art.

Controlled release vehicles are well known to those of skill in the pharmaceutical sciences. The technology and products in this art are variably referred to as controlled release, sustained release, prolonged action, depot, repository, delayed action, retarded release and timed release; the words “controlled release” as used herein is intended to incorporate each of the foregoing technologies.

Numerous controlled release vehicles are known, including biodegradable or bioerodable polymers such as polylactic acid, polyglycolic acid, and regenerated collagen. Known controlled release drug delivery devices include creams, lotions, tablets, capsules, gels, microspheres, liposomes, ocular inserts, minipumps, and other infusion devices such as pumps and syringes. Implantable or injectable polymer matrices, and transdermal formulations, from which active ingredients are slowly released are also well known and can be used in the disclosed methods.

In one embodiment, the compounds are administered through a topical delivery system. The controlled release components described above can be used as the means to delivery the disclosed compounds. A suitable topical delivery system comprises the disclosed compounds in concentrations taught herein, a solvent, an emulsifier, a pharmaceutically acceptable carrier material, penetration enhancing compounds, and preservatives. Examples of topically applied compositions include U.S. Pat. Nos. 5,716,610 and 5,804,203, which are hereby incorporated by reference. The compositions can further include components adapted to improve the stability or effectiveness of the applied formulation, such as preservatives, antioxidants, skin penetration enhancers and sustained release materials. Examples of such components are described in the following reference works hereby incorporated by reference: Martindale—The Extra Pharmacopoeia (Pharmaceutical Press, London 1993) and Martin (ed.), Remington's Pharmaceutical Sciences.

Controlled release preparations can be achieved by the use of polymers to complex or absorb the N-α-MH. The controlled delivery can be exercised by selecting an appropriate macromolecule such as polyesters, polyamino acids, polyvinylpyrrolidone, ethylenevinyl acetate, methylcellulose, carboxymethylcellulose, and protamine sulfate, and the concentration of these macromolecule as well as the methods of incorporation are selected in order to control release of active compound.

Hydrogels, wherein the N-α-MH is dissolved in an aqueous constituent to gradually release over time, can be prepared by copolymerization of hydrophilic mono-olefinic monomers such as ethylene glycol methacrylate. Matrix devices, wherein the ROS inhibiting or scavenging compound is dispersed in a matrix of carrier material, can be used. The carrier can be porous, non-porous, solid, semi-solid, permeable or impermeable. Alternatively, a device comprising a central reservoir of N-α-MH surrounded by a rate controlling membrane can be used to control the release of N-α-MH. Rate controlling membranes include ethylene-vinyl acetate copolymer or butylene terephthalate/polytetramethylene ether terephthalate. Use of silicon rubber depots are also contemplated.

Controlled release oral formulations are also well known. In one embodiment, the active compound is incorporated into a soluble or erodible matrix, such as a pill or a lozenge. Such formulations are well known in the art. An example of a lozenge used to administer pharmaceutically active compounds is U.S. Pat. No. 5,662,920, which is hereby incorporated by reference. In another example, the oral formulations can be a liquid used for sublingual administration. Examples of pharmaceutical compositions for liquid sublingual administration of the disclosed compounds are taught in U.S. Pat. No. 5,284,657, which is hereby incorporated by reference. These liquid compositions can also be in the form a gel or a paste. Hydrophilic gums, such as hydroxymethylcellulose, are commonly used. A lubricating agent such as magnesium stearate, stearic acid, or calcium stearate can be used to aid in the tableting process.

For the purpose of parenteral administration, N-α-MH can be combined with distilled water, preferably buffered to an appropriate pH and having appropriate (e.g., isotonic) salt concentrations. The compounds of the present invention can also be provided as a liquid or as a powder that is reconstituted before use. They can be provided as prepackaged vials, syringes, or injector systems.

N-α-MH can also be provided in septum-sealed vials in volumes ranging from about 0.5 to 100 ml for administration to an individual. In a preferred embodiment, the vials contain volumes of 0.5, 1, 3, 5, 6, 8, 10, 20, 50 and 100 ml. The vials are preferably sterile. The vials can optionally contain an isotonic carrier medium and/or a preservative. Any desired amount of N-α-MH can be used to give a desired final N-α-MH concentration. In a preferred embodiment, the N-α-MH concentration is between about 0.01 mg/ml and 100 mg/ml. More preferably, the N-α-MH concentration is between about 0.1 and 50 mg/ml. Most preferably, the N-α-MH concentration is between about 1 mg/ml and 10 mg/ml. At the lower end of the volume range, it is preferred that individual doses are administered, while at the higher end it is preferred that multiple doses are administered.

In a preferred embodiment, transdermal patches, steady state reservoirs sandwiched between an impervious backing and a membrane face, and transdermal formulations, can also be used to deliver N-α-MH. Transdermal administration systems are well known in the art. Occlusive transdermal patches for the administration of an active agent to the skin or mucosa are described in U.S. Pat. Nos. 4,573,996, 4,597,961 and 4,839,174, which are hereby incorporated by reference. One type of transdermal patch is a polymer matrix in which the active agent is dissolved in a polymer matrix through which the active ingredient diffuses to the skin. Such transdermal patches are disclosed in U.S. Pat. Nos. 4,839,174, 4,908,213 and 4,943,435, which are hereby incorporated by reference. In one embodiment, the steady state reservoir carries doses of N-α-MH in doses from about 0.2 to 5 mg per day.

Present transdermal patch systems are designed to deliver smaller doses over longer periods of time, up to days and weeks. A preferred delivery system for the disclosed compounds would specifically deliver an effective dose of N-α-MH in a range of between about 2 and 60 minutes, depending upon the dose, with a preferred dose being delivered within about 20-30 minutes. These patches allow rapid and controlled delivery of N-α-MH. A rate-controlling outer microporous membrane, or micropockets of the disclosed compounds dispersed throughout a silicone polymer matrix, can be used to control the release rate. Such rate-controlling means are described in U.S. Pat. No. 5,676,969, which is hereby incorporated by reference. In another preferred embodiment, N-α-MH is released from the patch into the skin of the patient in about 20-30 minutes or less. In a preferred embodiment, the compound is released from the patch at a rate of between about 0.025 mg to 0.5 mg per minute for a dose of between about 0.2 mg and 5 mg per patch.

These transdermal patches and formulations can be used with or without use of a penetration enhancer such as dimethylsulfoxide (DMSO), combinations of sucrose fatty acid esters with a sulfoxide or phosphoric oxide, or eugenol. The use of electrolytic transdermal patches is also within the scope of the methods disclosed herein. Electrolytic transdermal patches are described in U.S. Pat. Nos. 5,474,527, 5,336,168, and 5,328,454, the entire contents of which are hereby incorporated by reference.

In another embodiment transmucosal patches can be used to administer the disclosed compounds. An example of such a patch is found in U.S. Pat. No. 5,122,127, which is hereby incorporated by reference. The described patch comprises a housing capable of enclosing a quantity of therapeutic agent where the housing is capable of adhering to mucosal tissues, for example, in the mouth. A drug surface area of the device is present for contacting the mucosal tissues of the host. The device is designed to deliver the drug in proportion to the size of the drug/mucosa interface. Accordingly, drug delivery rates can be adjusted by altering the size of the contact area.

The housing is preferably constructed of a material which is nontoxic, chemically stable, and non-reactive with the disclosed compounds. Possible construction materials include: polyethylene, polyolefins, polyamides, polycarbonates, vinyl polymers, and other similar materials known in the art. The housing can contain means for maintaining the housing positioned against the mucosal membrane. The housing can contain a steady state reservoir positioned to be in fluid contact with mucosal tissue.

Steady state reservoirs for use with the disclosed compounds allow the delivery a suitable dose of those compounds over a predetermined period of time. Compositions and methods of manufacturing compositions capable of absorption through the mucosal tissues are taught in U.S. Pat. No. 5,288,497, which is hereby incorporated by reference. One of skill in the art could readily include the disclosed compounds and related compositions.

The steady state reservoirs for use with the disclosed compounds are composed of compounds known in the art to control the rate of drug release. In one embodiment, the transmucosal patch delivers a dose of N-α-MH over a period of time from about 2 to 60 minutes. The steady state reservoir contained within the housing carries doses of N-α-MH in doses from about 0.2 to 100 mg per patch. Transdermal patches that can be worn for several days and that release the disclosed compounds over that period of time are also contemplated. The reservoirs can also contain permeation or penetration enhancers, as discussed above, to improve the permeability of the disclosed compounds across the mucosal tissue.

Another method to control the release of the disclosed compounds is to incorporate the N-α-MH into particles of a polymeric material such as polyesters, polyamino acids, hydrogels, poly lactic acid, or ethylene vinylacetate copolymers.

Alternatively, instead of incorporating the N-α-MH into these polymeric particles, the disclosed compounds are entrapped in microcapsules prepared, for example, by coacervation techniques, or by interfacial polymerization, for example hydroxymethylcellulose or gelatin-microcapsules, respectively, or in colloidal drug delivery systems, for example, liposomes, albumin microspheres, microemulsions, nanoparticles, and nanocapsules, or in macroemulsions. Such technology is well known to those of ordinary skill in pharmaceutical sciences.

Preferably, the N-α-MH is injected, infused, or released into the patient at a rate of from about 0.025 to 1.0 mg/min. A rate of about 0.1 mg/min is preferred. The disclosed compounds are preferably administered over a period of time ranging from about 1, 3 or 5 minutes to about 30 minutes, with an upper limit of about 20 minutes being preferred, such that the total daily adult dose of N-α-MH ranges from between about 0.1 to about 100.0 mg, with about 0.1 to about 20.0 mg being preferred.

In another embodiment, N-α-MH at approximately 0.2 to 2.0 mg or 3-200 μg/kg, in a pharmaceutically acceptable form can be administered. ROS scavenging compounds can also be administered in combination with N-α-MH. When the N-α-MH is administered orally, the composition can be formulated as a tablet comprising between 10 mg to 2 grams of active ingredient. A tablet can include 10, 20, 50, 100, 200, 500, 1,000, or 2,000 milligrams [of N-α-MH. Preferably, the amount of N-α-MH in a tablet is 100 mg. In some embodiments, the composition includes histamine protectors such as diamine oxidase inhibitors, monoamine oxidase inhibitors and n-methyl transferases.

The treatment can also include periodically boosting patient blood N-α-MH levels by administering 0.2 to 2.0 mg or 3-200 μg/kg of the disclosed compounds injected or ingested 1, 2, or more times per day over a period of one to two weeks at regular intervals, such as daily, bi-weekly, or weekly in order to establish blood levels of N-α-MH at a beneficial concentration such that ROS production and release is inhibited. The treatment is continued until the causes of the patient's underlying disease state is controlled or eliminated.

Administration of each dose of N-α-MH can occur from once a day to up to about four times a day, with twice a day being preferred. Administration can be subcutaneous, intravenous, intramuscular, intraocular, oral, transdermal, intranasal, or rectal and can utilize direct hypodermic or other injection or infusion means, or can be mediated by a controlled release mechanism of the type disclosed above. Any controlled release vehicle or infusion device capable of administering a therapeutically effective amount of the disclosed compounds over a period of time ranging from about 1 to about 90 minutes can be used. In a preferred embodiment, intranasal delivery is accomplished by using a solution of N-α-MH in an atomizer or nebulizer to produce a fine mist which is introduced into the nostrils. For rectal delivery, N-α-MH is formulated into a suppository using methods well known in the art.

In some embodiments, N-α-MH is administered with a compound that scavenge ROS. ROS scavengers can be administered in an amount of from about 0.1 to about 20 mg/day; more preferably, the amount is from about 0.5 to about 8 mg/day; more preferably, the amount is from about 0.5 to about 8 mg/day; and even more preferably, the amount is from about 1 to about 5 mg/day. Nevertheless, in each case, the dose depends on the activity of the administered compound. The foregoing doses are appropriate for the enzymes listed above that include catalase, superoxide dismutase (SOD), glutathione peroxidase and ascorbate peroxidase. Appropriate doses for any particular host can be readily determined by empirical techniques well known to those of ordinary skill in the art.

Non-enzymatic ROS scavengers can be administered in amounts empirically determined by one of ordinary skill in the art. For example, vitamins A and E can be administered in doses from about 1 to 5000 IU per day. Vitamin C can be administered in doses from about 1 μg to 10 gm per day. Minerals such as selenium and manganese can be administered in amounts from about 1 picogram to 1 milligram per day. These compounds can also be administered as a protective or preventive treatment for ROS mediated disease states.

In addition to N-α-MH, certain embodiments include the coadministration of a compound which induces the release of endogenous histamine stores. Retinoic acid, other retinoids such as 9-cis-retinoic acid and all-trans-retinoic acid, IL-3 and ingestible allergens are compounds that are known to induce the release of endogenous histamine. These compounds can be administered to the patient by oral, intravenous, intramuscular, subcutaneous, and other approved routes. The rate of administration should result in a release of endogenous histamine resulting in a blood plasma level of histamine of about 20 nmol/dl.

Administration of each dose of a compound which induces histamine release can occur from once per day to up to about four times a day, with twice per day being preferred. Administration can be subcutaneous, intravenous, intramuscular, intraocular, oral, or transdermal, and can incorporate a controlled release mechanism of the type disclosed above. Any controlled release vehicle capable of administering a therapeutically effective amount of a compound which induces histamine release over a period of time ranging from about one to about thirty minutes can be used. Additionally, the compounds, compositions, and formulations of the present invention can be administered quantum sufficiat.

The following examples teach the unexpectedly superior results of the administration of N-alpha-methyl-histamine dihydrochloride (N-α-MH) as compared with histamine dihydrochloride. These examples are illustrative only and are not intended to limit the scope of the claims. The treatment methods described below can be optimized using empirical techniques well known to those of ordinary skill in the art. Moreover, artisans of ordinary skill would be able to use the teachings described in the following examples to practice the full scope of the claims.

### Example 1

The effects of histamine dihydrochloride (HDC) and N-α-MH on oxygen radical formation by human mononuclear phagocytes in response to N-formylmethionyl-leucyl-phenylalanine (fMLF) were evaluated. Mononuclear phagocytes were isolated and oxygen radical formation was measured as described in Hellstrand et al., J. Immunology, vol. 153, pp 4940-7, hereby incorporated by reference in its entirety. HDC or N-α-MH were preincubated with cells for five minutes followed by the addition of fMLF and then oxygen radical production was assessed.

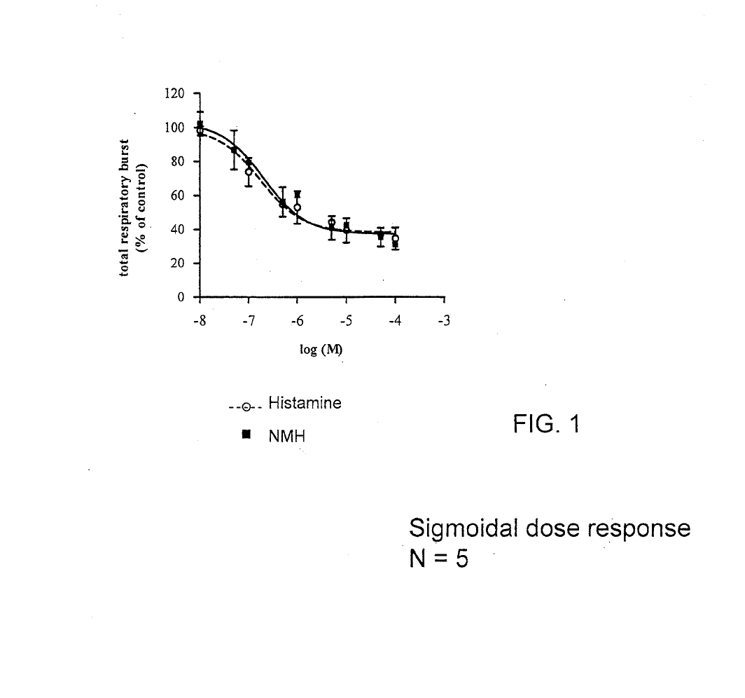

FIG. 1 illustrates the results of experiments performed to assess the effect of N-α-MH on oxygen radicals (superanion production) in human mononuclear phagocytes. The results indicate that N-α-MH is a potent inhibitor of oxygen radical production. The maximal response in terms of oxygen radical inhibition of N-α-MH is at least comparable to HDC in vitro as illustrated in FIG. 1. As will be described below, N-α-MH was approximately 10 times more potent in reducing tumor formation in vivo when compared with HDC.

### Example 2

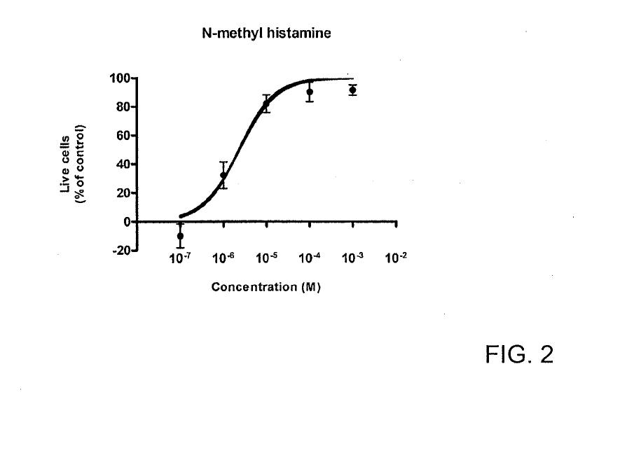

The ability of N-α-MH to protect NK cells from apoptosis inflicted by mononuclear phagocytes was evaluated. Cells were prepared as described by Hellstrand et al., J. Immunol., vol. 153, pp 4940-7, and mixtures of mononuclear phagocytes and NK cells were incubated overnight with HDC or N-α-MH at indicated final concentrations in 96-well microplates. Thereafter, cells were recovered and NK cells were analyzed for apoptosis (forward and side scatter) as described in Betten et al., J. Clin. Invest. Vol. 108, pp 1221-8 (hereby incorporated by reference in its entirety). FIG. 2 plots the results of six similar experiments.

The ED₅₀ values for protection of NK cells were 2.2 μM for N-α-MH. The fact that the ED₅₀ value was slightly higher than in the oxygen radical burst experiment described in Example 1 is likely attributable to consumption of the compound during the overnight assay.

Histamine dihydrochloride protects NK cells from apoptosis by inhibiting the release of oxygen radicals from mononuclear phagocytes. See, Hellstrand et al., JI 1994. The data reflected in FIGS. 1 and 2 support the finding that N-α-MH is a comparable inhibitor of oxygen radical production to HDC and is also equally potent and effective at protecting NK cells from apoptosis as HDC in vitro.

### Example 3

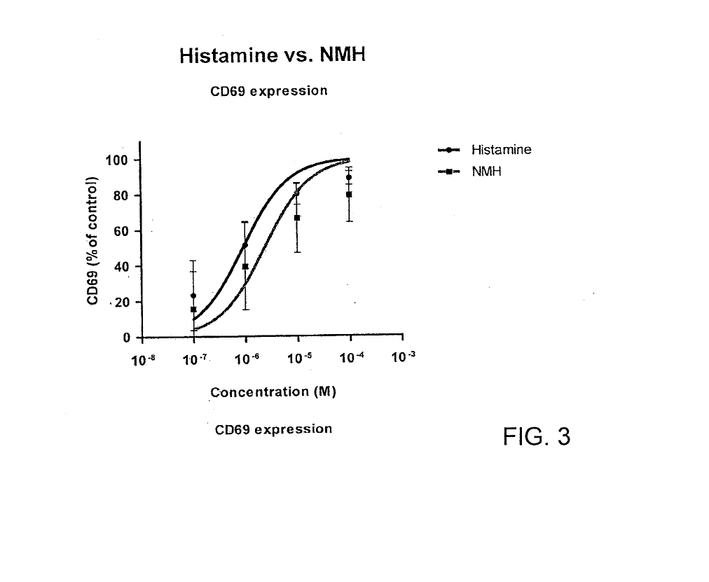

Three separate experiments were performed as described above with the addition of human recombinant IL-2 (50 U/ml) during the overnight incubation. The experiments were initiated with the aim at clarifying whether N-α-MH synergizes with IL-2 to activate NK cells in a fashion similar to HDC. The activation of NK cells by IL-2 was measured as the capacity of these cells to express the cell surface activation antigen CD69 (See, generally Hellstrand et al. JI 1994). The results of these experiments are demonstrated in FIG. 3.

As illustrated in FIG. 3, N-α-MH, at concentrations similar to those required for oxygen radical inhibition and NK cell protection, synergized with IL-2 to induce expression of CD69 on NK cells.

When compared with HDC, N-α-MH is as potent and efficacious in inhibiting oxygen radical production. N-α-MH also protects human NK cells and synergizes with IL-2 to activate NK cells.

### Example 4

The anti-tumor properties of N-α-MH on i.v. injected B16 melanoma cells in mice were evaluated. One of N-α-MH, HDC, and a positive control were administered to the mice 24 hours before tumor cell inoculation as described by Hellstrand et al., J. Immunol., vol. 145, pp 4365-4370. Macroscopically visible pulmonary metastatic foci were counted fourteen days after the tumor cell inoculation. The tumor weight is the wet weight at day 14 of lungs from mice injected with tumor cells on day 0 as described by Hellstrand et al., J. Immunol., vol. 145, pp 4365-4370.

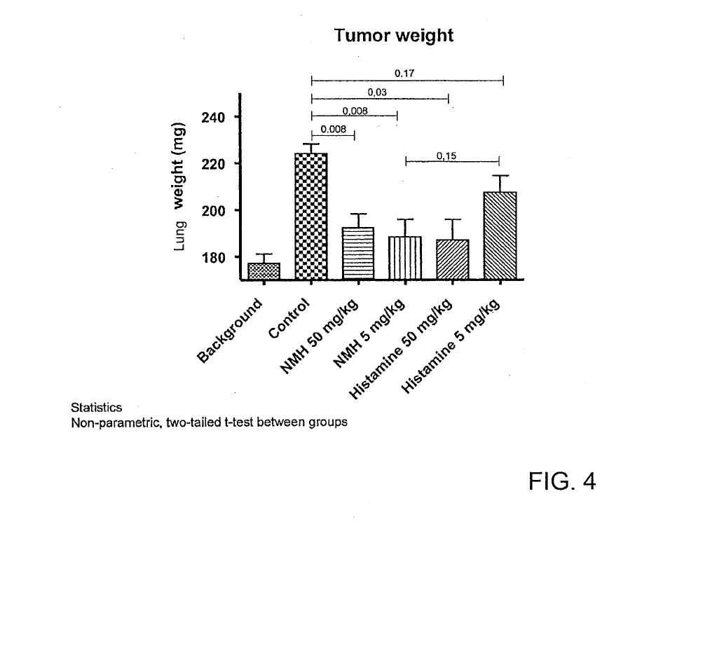

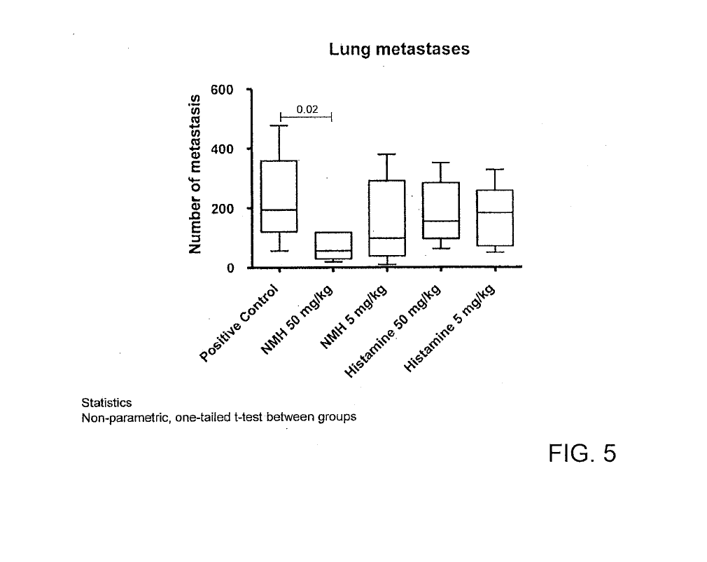

FIGS. 4 and 5 illustrate the results (expressed as mean+/−s.e.m.) of these experiments. FIG. 4 depicts the tumor weight. Mice were administered high or low doses of N-α-MH or high or low doses of HDC. The lung weight of tumor-free mice is shown for reference (Background). The Control bar shows the weight of corresponding lungs from mice injected with B16 melanoma cells on day 0, with lung weight measured 14 days later. As shown, both N-α-MH and HDC at 50 mg/kg significantly reduced tumor weight when compared with control mice. In contrast to HDC, N-α-MH significantly reduced tumor weight also at 5 mg/kg. The extent of tumor weight reduction achieved by N-α-MH at 5 mg/kg was equal to that achieved by histamine at 50 mg/kg, which implies that N-α-MH is about 10 times more potent than HDC in reducing tumor weight in vivo.

FIG. 5 is a box plot depicting the 10^{th}, 25^{th}, 50^{th}, 75^{th}, and 90^{th} percentiles of the number of melanoma lung metastases visibly detected on the surface of mouse lungs 14 days after tumor cell inoculation. The Positive Control bar shows the number of visible lung metastases in tumor cell-injected mice. Mice were administered high or low doses of N-α-MH or high or low doses of HDC. Mice receiving 50 mg/kg of N-α-MH demonstrated significantly less lung metastases than control mice. The extent of tumor reduction was about equal in mice treated with 5 mg/kg of N-α-MH when compared with mice treated with HDC at 50 mg/kg. These results imply that N-α-MH is approximately 10 times more potent in reducing melanoma metastasis in vivo than HDC, and are thus consistent with the results presented in FIG. 4.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being redefined herein to be restricted to including any specific characteristics of the features or aspects of the invention with which that terminology is associated. The scope of the invention should therefore be construed in accordance with the appended claims and any equivalents thereof.

1. An improved method of augmenting NK cell cytotoxicity with a histamine metabolite while reducing untoward side effects associated with histamine administration, comprising administering:
a first composition comprising a pharmaceutically effective amount of N-alpha-methylhistamine dihydrochloride; and
a second composition comprising a cytokine.

2. The method of claim 1, wherein said cytokine is interleukin-2.

3. The method of claim 1, wherein said cytokine is Interferon-α, Interferon-β, Interferon-γ, or combinations thereof.

4. The method of claim 1, wherein said cytokine is selected from the group consisting of IL-1, IL-3, IL-12, and IL-15.

5. The method of claim 1, wherein said effective amount of N-alpha-methylhistamine dihydrochloride is between about 0.005 and 10 mg/kg/day.

6. The method of claim 1, wherein said Interleukin-2 (IL-2) is administered in an amount of between about 5,000 and 300,000 U/kg/day.

7. A method of enhancing the NK cell response to IL-2, comprising administering to a population of NK cells:
a first composition comprising a pharmaceutically effective amount of IL-2; and
a second composition comprising a pharmaceutically effective amount of N-alpha-methyl-histamine dihydrochloride.

8. The method of claim 7, wherein said pharmaceutically effective amount of IL-2 is between about 5,000 and 300,000 U/kg/day.

9. The method of claim 7, wherein said pharmaceutically effective amount of N-alpha-methyl-histamine dihydrochloride is between about 0.005 and 10 mg/kg/day.

10. A method of treating a condition caused or exacerbated by oxygen radical formation, comprising:
identifying an individual suffering from a condition caused or exacerbated by oxygen radical formation; and
administering to said individual a pharmaceutically effective amount of N-alpha-methyl-histamine dihydrochloride.

11. The method of claim 10, wherein said condition is selected from the group consisting of viral infection, autoimmune disease, inflammatory disease, atherosclerosis, cancer, and neurodegenerative disease.

12. The method of claim 10, wherein said effective amount of N-alpha-methyl-histamine dihydrochloride is between about 0.005 and 10 mg/kg/day.

13. A method of protecting NK cells from oxidative damage inflicted by oxygen radicals in a subject, for the treatment of cancer, viral diseases, or inflammatory diseases, said method comprising:
identifying a subject in need of NK cell enhancement; and
administering to said subject an effective amount of N-alpha-methyl-histamine dihydrochloride.

14. The method of claim 13, further comprising administering a cytokine selected from the group consisting of IL-1, IL-2, IL3, IL12, IL-15, IFN-α, IFN-β, and IFN-γ.

15. The use of N-alpha-methylhistamine dihydrochloride for the treatment of a condition caused or exacerbated by oxygen free radical formation.

16. The use of claim 15, wherein the condition is selected from the group consisting of viral infection, autoimmune disease, inflammatory disease, atherosclerosis, cancer, and neurodegenerative disease.

17. A compound comprising:
a pharmaceutically effective amount of N-alpha-methylhistamine dihydrochloride; and
a cytokine.
